SEQUENCE LISTING (4 sequences)

SEQ ID NO: 1 - DNA, 21 bp, ARTIFICIAL SEQUENCE
  SYNTHETIC DNA
agtgtttgtggtgggtggaga

SEQ ID NO: 2 - DNA, 21 bp, ARTIFICIAL SEQUENCE
  SYNTHETIC DNA
ttatgaaacatactgagctcc

SEQ ID NO: 3 - DNA, 23 bp, ARTIFICIAL SEQUENCE
  SYNTHETIC DNA
acaactttggtatcgtggaagga

SEQ ID NO: 4 - DNA, 22 bp, ARTIFICIAL SEQUENCE
  SYNTHETIC DNA
ccgttcagctcagggatgacct

(19) United States
(12) Patent Application Publication
(10) Pub. No.: US 2008/0161348 A1
(43) Pub. Date: Jul. 3, 2008

(54) PTX3-GENE EXPRESSION INHIBITOR

(75) Inventors: Shigeru MORIKAWA, Tokyo (JP); Akashi Izumi, Tokyo (JP); Takao Hamakubo, Tokyo (JP); Tatsuhiko Kodama, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KOWA CO., LTD., Nagoya-shi (JP); Nissan Chemical Industries, Ltd., TOKYO (JP)

(21) Appl. No.: 11/694,288

(22) Filed: Mar. 30, 2007

Related U.S. Application Data
(63) Continuation of application No. 10/196,428, filed on Jul. 17, 2002, now abandoned.
(60) Provisional application No. 60/372,114, filed on Apr. 15, 2002.

(51) Int. Cl. A61K 31/695; A61K 31/47

(52) U.S. Cl. 514/311; 514/300; 514/63; 514/423; 514/548; 514/406; 514/415; 514/303; 514/301; 514/422

(57) ABSTRACT

A method for suppressing expression of PTX3 gene, which comprises administering an effective amount of a compound, which is represented by the following formula (1):

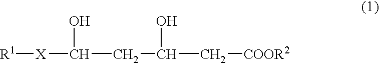

wherein R¹ represents an organic group, X represents —CH₂CH₂— or —CH═CH—, and R² represents a hydrogen atom or an alkyl group, or a lactone derivative thereof, or a salt thereof.

According to the present invention, a PTX3 gene expression suppressing method useful for the treatment of autoimmune diseases, especially rheumatoid arthritis can be provided.

### TECHNICAL FIELD

This invention relates to a pentraxin 3 (PTX 3) gene expression suppressing method useful for the treatment of autoimmune diseases, especially rheumatoid arthritis.

### BACKGROUND ART

PTX3 gene was found as a novel gene the expression of which is induced by interleukin-1 (IL-1) from normal human umbilical vein endothelial cells (HUVEC) [Breviario et al.: J. Biol. Chem., 267(31), 22190-7 (1992)]. Further, a gene (TSG-14 gene) the expression of which is induced by tumor necrosis factor α (TNF-α) from human fibroblasts was also found [Lee et al.: J. Immunol., 150(5), 1804-12 (1993)], and from a structural analysis, this gene has been found to be the same as PTX3 gene. PTX3 protein, in view of its molecular structure, belongs to the so-called pentraxin family such as C-reactive protein (CRP) and serum amyloid P component (SAP), but its physiological functions are not known much. For reasons such that PTX3 protein is not induced by IL-6 and is different from the species of cells to be produced, PTX3 protein was suggested to have functions different from CRP or SAP [J. Biol. Chem., 267(31), 22190-7 (1992); Domyaku Koka (Arteriosclerosis), 24(7-8), 375-80 (1996)].

As relevancy to the inflammatory reaction such as a formation of an arteriosclerotic layer or an ischemic heart disease, it has been found that the blood level of PTX3 is high in acute myocardial infarction patients [Circulation, 102, 636-41 (2000)] and that expression of a tissue factor, an important factor for the formation of thrombus, is increased by PTX3 [Arterioscler. Thromb. Vasc. Biol., 22, 782-7 (2002)].

Recently, it has also been revealed that PTX3 gene is constantly expressed in synovial cells of a rheumatoid arthritis patient and that this expression is suppressed by inteferon-γ (IFN-γ) or transforming growth factor-β (TGF-β) [Clin. Exp. Immunol., 119(1), 196-202 (2000)]. Moreover, PTX3 also takes part in a disorder via a complement pathway in an autoimmune disease, especially rheumatoid arthritis, because it binds to Clq, one of complement components, to activate the complement pathway [J. Biol. Chem., 272(52), 32817-23 (1997)].

Suppression of PTX3 gene expression, therefore, suppresses worsening of an autoimmune disease, especially rheumatoid arthritis and further, results in its treatment. Except for IFN-γ and TGF-β, however, absolutely no substance has been known to date to suppress expression of PTX3 gene.

An object of the present invention is, therefore, to provide a PTX3 gene expression suppressing method, which suppresses expression of PTX3 gene and is effective for the treatment of an autoimmune disease, especially rheumatoid arthritis.

### DISCLOSURE OF THE INVENTION

Using a cultured human cell system, the present inventors have hence looked for substances which affect expression of PTX3 gene. As a result, it has been quite unexpectedly found that compounds represented by the below-described general formula (1) and their lactone derivatives and salts of these compounds and lactone derivatives, all of which are known as HMG-CoA reductase suppressors, especially pitavastatin calcium and atorvastatin calcium have activities to suppress expression of PTX3 gene, leading to the completion of the present invention.

Described specifically, the present invention provides a method for suppressing expression of PTX3 gene, which comprises administering an effective amount of a compound, which is represented by the following formula (1):

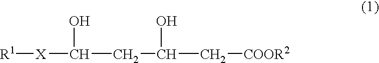

wherein R¹ represents an organic group, X represents —CH₂CH₂— or —CH═CH—, and R² represents a hydrogen atom or an alkyl group, or a lactone derivative thereof, or a salt thereof, as an active ingredient.

### BRIEF DESCRIPTION OF THE DRAWINGS

FIG. 1 is a diagram showing expression levels of PTX3 gene; and

FIG. 2 is a diagram electrophoretically illustrating suppression of gene expression.

### BEST MODES FOR CARRYING OUT THE INVENTION

Compounds represented by the formula (1), their lactone derivatives and salts of these compounds and lactone derivatives, all of which are usable in the present invention, are known as HMG-CoA reductase suppressors useful as hyperlipidemia therapeutics. However, absolutely nothing is known as to whether or not they affect expression of PTX3 gene.

The organic group represented by R¹ in the compound represented by the formula (1) may preferably be a substituted or unsubstituted organic group having a cyclic structure.

Examples of the organic group having the cyclic structure can include indolyl, indenyl, pyridyl, pyrrolopyridyl, pyrazolopyridyl, thienopyridyl, pyrimidyl, pyrazolyl, pyrrolyl, imidazolyl, indolidyl, quinolyl, naphthyl, hexahydronaphthyl, cyclohexyl, phenylsilylphenyl, phenylthienyl and phenylfuryl groups, with hexahydronaphthyl, indolyl, pyridyl, pyrimidyl, pyrrolyl and quinolyl groups being particularly preferred.

Examples of substituent groups, which may substitute on these organic groups having the cyclic structures, can include hydroxyl group, linear, branched or cyclic alkyl groups, alkyloxyalkyl groups, alkylcarbonyloxy groups, alkyl-substituted amino groups, substituted alkylsulfonylamino groups, substituted phenylsulfonylamino groups, carbamoyl group which may be substituted by one or two alkyl or phenyl groups, halophenyl groups, alkylphenyl groups, alkoxyphenyl groups, phenyl group, and oxo group.

Among these substituents which may substitute on these organic groups having the cyclic structures, preferred are linear, branched or cyclic C₁₋₆ alkyl groups, C₂₋₇ alkyloxyalkyl groups, C₁₋₄ acyloxy groups, C₁₋₄ alkyl-substituted amino groups, C₁₋₄ alkyl-substituted C₁₋₄ alkylsulfonylamino groups, C₁₋₄ alkyl-substituted phenylsulfonylamino groups, C₁₋₄ alkyl-substituted carbamoyl groups, phenyl-substituted carbamoyl groups, fluorophenyl groups, bromophenyl groups, iodophenyl groups, methylphenyl groups, ethylphenyl groups, metoxyphenyl groups, ethoxyphenyl groups and phenyl group, with isopropyl, cyclopropyl and p-fluorophenyl groups being particularly preferred.

Examples of the alkyl group represented by R² may include a linear, branched or cyclic alkyl group having 1-6 carbon atoms.

Examples of the alkyl group represented by R² may include a linear, branched or cyclic alkyl group having 1-6 carbon atoms.

The lactone derivative can be obtained by subjecting its corresponding compound, which is represented by the formula (1), to lactonization in a manner known per se in the art, for example, under acidic conditions.

The salts of the compound represented by the formula (1) and its lactone derivative are physiologically acceptable salts. Examples can include alkali metal salts such as the sodium salts and potassium salts, alkaline earth metal salts such as the calcium salts and magnesium salts, organic amine salts such as the phenethylamine salts, and the ammonium salts, with the sodium salts and calcium salts being more preferred.

These compounds are disclosed, for example, in U.S. Pat. No. 4,739,073 and EP-A-114,027; EP-A-367,895; U.S. Pat. No. 5,001,255, U.S. Pat. No. 4,613,610, U.S. Pat. No. 4,851,427, U.S. Pat. No. 4,755,606, U.S. Pat. No. 4,808,607, U.S. Pat. No. 4,751,235, U.S. Pat. No. 4,939,159, U.S. Pat. No. 4,822,799, U.S. Pat. No. 4,804,679, U.S. Pat. No. 4,876,280, U.S. Pat. No. 4,829,081, U.S. Pat. No. 4,927,851, U.S. Pat. No. 4,588,715; F. G. Kathawala, Medical Research Reviews, 11, 121-146 (1991), EP-A-304,063; EP-A-330,057; U.S. Pat. No. 5,026,708, U.S. Pat. No. 4,868,185; EP-A-324,347; EP-A-300,278; U.S. Pat. No. 5,013,749, U.S. Pat. No. 5,872,130, U.S. Pat. No. 5,856,336, U.S. Pat. No. 4,231,938, U.S. Pat. No. 4,444,784, U.S. Pat. No. 4,346,227, U.S. Pat. No. 5,354,772, U.S. Pat. No. 5,273,995, U.S. Pat. No. 5,177,080, U.S. Pat. No. 3,983,140, JP-B-2,648,897, U.S. Pat. No. 5,260,440, Bioorganic & Medicinal Chemistry, 5, 437 (1977), JP-B-2,569,746, EP-B-304,063, and U.S. Pat. No. 5,856,336.

Preferred examples of the active ingredient in the method according to the present invention for the suppression of expression of PTX3 gene can include lovastatin (U.S. Pat. No. 4,231,938: (+)-(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthyl (S)-2-methylbutyrate), simvastatin (U.S. Pat. No. 4,444,784: (+)-(1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthyl 2,2-dimethylbutanoate), pravastatin (U.S. Pat. No. 4,346,227: (+)-(3R,5R)-3,5-dihydroxy-7-[(1S,2S,6S,8S,8aR)-6-hydroxy-2-methyl-8-[(S)-2-methylbutyryloxy]-1,2,6,7,8,8a-hexahydro-1-naphthyl]heptanoic acid), fluvastatin (U.S. Pat. No. 5,354,772: (3RS,5SR,6E)-7-[3-(4-fluorophenyl)-1-(1-methylethyl)-1H-indol-2-yl]-3,5-dihydroxy-6-heptenoic acid), atorvastatin (U.S. Pat. No. 5,273,995: (3R,5R)-7-[2-(4-fluorophenyl)-5-isopropyl-3-phenyl-4-phenylcarbamonyl-1H-pyrrol-1-yl]-3,5-dihydroxyheptanoic acid), cerivastatin (U.S. Pat. No. 5,177,080: (3R,5S)-erythro-(E)-7-[4-(4-fluorophenyl)-2,6-diisopropyl-5-methoxymethyl-pyridin-3-yl]-3,5-dihydroxy-6-heptenoic acid), mevastatin (U.S. Pat. No. 3,983,140: (+)-(1S,3R,7S,8S,8aR)-1,2,3,7,8,8a-hexahydro-7-methyl-8-[2-[(2R,4R)-tetrahydro-4-hydroxy-6-oxo-2H-pyran-2-yl]ethyl]-1-naphthyl(S)-2-methylbutyrate), rosuvastatin (U.S. Pat. No. 5,260,440, JP-B-2,648,897: 7-[4-(4-fluorophenyl)-6-isopropyl-2-(N-methyl-N-methanesulfonylaminopyrimidin)-5-yl]-(3R,5S)-dihydroxy-(E)-6-heptenoic acid), and pitavastatin (U.S. Pat. No. 5,856,336, JP-B-2,569,746: (3R,5S, 6 E)-7-[2-cyclopropyl-4-(4-fluorophenyl)-3-quinolyl]-3,5-dihydroxy-6-heptenoic acid, and their salts. In particular, pitavastatin and its salts and atorvastatin and its salts are preferred.

The compound represented by the formula (1) and its lactone derivative and the salts of these compound and lactone derivatives, all of which are useful in the present invention, significantly suppress expression of mRNA for PTX3 in human cells and therefore, are useful in the PTX3 gene expression suppressing method according to the present invention, especially for the treatment of autoimmune diseases such as rheumatoid arthritis. Further, they also permit inter alia development of experiment systems, in which PTX3 takes part, and screening of novel medicines.

Illustrative administration routes for the compound (1) or its lactone or the salt of the compound or lactone can include oral administrations by tablets, capsules, a granule, a powder, a syrup and the like; and parenteral administrations by an intravenous injection, an intramuscular injection, suppositories, an inhalant, a transdermal preparation, an eye drop, a nasal drop and the like.

To formulate medicinal preparations in such various forms as described above, the active ingredient can be used either singly or in combination with one or more of pharmaceutically acceptable excipients, binders, extenders, disintegrants, surfactants, lubricants, dispersants, buffering agents, preservatives, corrigents, perfumes, coating materials, carriers, diluents and the like, as needed.

Of these administration routes, oral administrations are preferred. Upon formulation of a medicinal preparation for oral administration, it is preferred to adjust the pH in view of the stability of the active ingredient (JP-A-2-0006406, JP-B-2,774,037, WO-A-97/23200, etc.).

The dosage of the active ingredient varies inter alia depending on the weight, age, sex and conditions of each patient. In the case of an adult, however, it is generally preferred to orally or parenterally administer the active ingredient at a daily dosage of from 0.01 to 1,000 mg, specifically from 0.1 to 100 mg in terms of the compound represented by formula (1) at once or in several portions.

### EXAMPLES

The present invention will hereinafter be described in detail based on Examples. It should however be borne in mind that the present invention is not limited to the following Examples.

### Example 1

Two days after inoculation of normal human umbilical vein endothelial cells (HUVEC) or human coronary artery smooth muscle cells (HCASMC) at 3×10⁵ cells/10 cm dish, pitavastatin calcium or atorvastatin calcium was added to 1.1 μmol/L and 6.6 μmol/L, respectively. Dimethyl sulfoxide, a solvent for both of the active ingredients, was added to a control (final concentration: 0.0066 v/v %). Eight hours after the addition, total RNA was extracted with “ISOGEN” (trade mark, product of NIPPON GENE CO., LTD.). The following procedures was conducted in accordance with the procedures manual of Affymetrix. Inc. Described specifically, mRNA was isolated from the above-obtained total RNA, and based on the mRNA, cDNA was synthesized. Further, biotin-labeled cRNA was synthesized by in vitro transcription. Subsequent to purification, the biotin-labeled cRNA was subjected to fragmentation by heat treatment to prepare fragmented cRNA for use in a gene expression analysis.

Gene expression analysis method: The fragmented CRNA was poured into “Hugene Human FL Array” (trade name, product of Affymetrix, Inc.), and hybridization was conducted at 45° C. for 16 hours. Subsequent to washing, staining with phycoerythrin-labeled streptavidin and biotinylated antistreptavidin antibody was applied, and gene expression information was inputted by “GeneChip™ Scanner” (trade name, manufactured by Hewlett Packard Company). The information was analyzed by “GENECHIP SOFTWARE” (trade name, product of Affymetrix, Inc.) to compare expression levels.

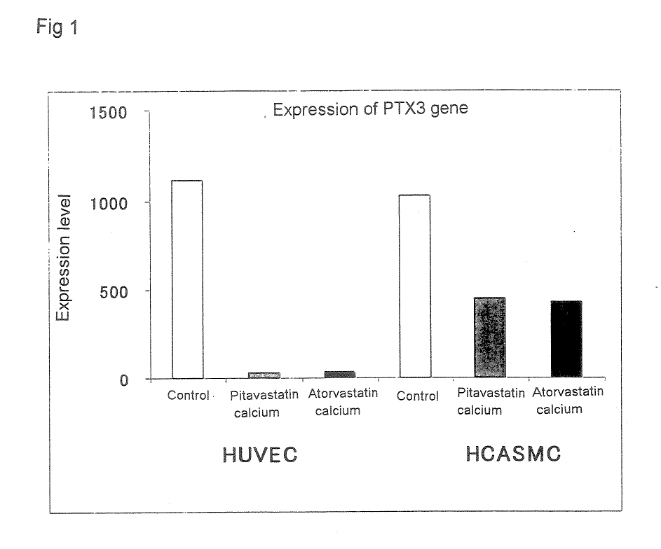

The results of the measurement are shown in FIG. 1.

The expression of PTX3 gene in HUVEC upon elapsed time of 8 hours after the addition of the active ingredient was significantly suppressed to 32.7 and 39.2 in the pitavastatin calcium and atorvastatin calcium addition groups, respectively, as opposed to 1113.0 in the control. The expression of PTX3 gene in HCASMC upon elapsed time of 8 hours after the addition of the active ingredient was also significantly suppressed to 452.5 and 432.1 in the pitavastatin calcium and atorvastatin calcium addition groups, respectively, as opposed to 1028.3 in the control.

### Example 2

Two days after inoculation of HUVEC at 3×10⁵ cells/10 cm dish, pitavastatin calcium or atorvastatin calcium was added to 1.1 μmol/L and 6.6 μmol/L, respectively. To ascertain possible concentration dependency of PTX3 gene expression suppressing effect of pitavastatin, pitavastatin calcium was also added to 1 μmol/L and 10 μmol/L upon elapsed time of 2 days after inoculation of HUCEC or HCASMC at 3×10⁵ cells/10 cm dish. To controls under the respective conditions, dimethyl sulfoxide, a solvent for both of the active ingredients, was added (final concentration: 0.0066 v/v %) Eight or 24 hours after the addition, total RNA was extracted with “ISOGEN” (trade mark, product of NIPPON GENE CO., LTD.). The total RNA was subjected to RT-PCR in a manner known per se in the art, and amplified DNA fragments were subjected to agarose gel electrophoresis to compare expression levels.
Reaction conditions and the like for PT-PCR:

PT reaction: Conducted using “RNA PCR Core Kit” (trade name, product of Roche Molecular Systems, Inc.).

PCR: Using “Expanded™ High Fidelity PCR System” (trade name, manufactured of Boehringer Mannheim AG), thermal cycling was conducted through 25 cycles according to the following schemes: 95° C. for 1 minute—57° C. for 1 minute—72° C. for 1 minute. Incidentally, as PCR primers, the followings were used in sets: SEQ ID No:1 (Forward) and SEQ ID No: 2 (Reverse) in the case of PTX3; base SEQ ID No:3 (Forward) and SEQ ID No: 4 (Reverse) in the case of GAPDH.

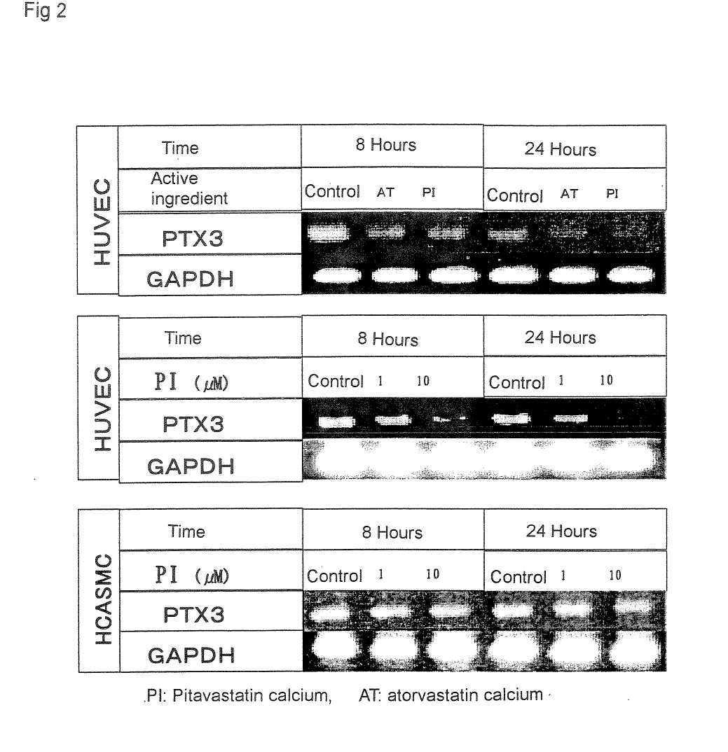

The results are shown in FIG. 2.

The expression of PTX3 gene in HUVEC was suppressed by the addition of pitavastatin calcium or atorvastatin calcium both 8 hours later and 24 hours later compared with the control. Further, the expressions of PTX3 gene in HUVEC and HCASMC were concentration-dependently suppressed by the addition of pitavastatin calcium both 8 hours later and 24 hours later.

### INDUSTRIAL APPLICABILITY

The present invention can provide a PTX3 gene expression suppressing method useful for the treatment of autoimmune diseases, especially rheumatoid arthritis

1-15. (canceled)

16. A method for suppressing expression of PTX3 gene, which comprises administering to a subject in need thereof an effective amount of a composition comprising pitavastatin, or a salt thereof, as an active ingredient, wherein said subject in need thereof suffers from rheumatoid arthritis.

17. The method according to claim 16, wherein said administering is oral administration.

18. The method according to claim 17, wherein said oral administration comprises administering said compound of formula (1) in a form selected from the group consisting of a tablet, a capsule, a granule, a powder, and a syrup.

19. The method according to claim 16, wherein said administering is parenteral administration.

20. The method according to claim 19, wherein said parenteral administration comprises administering said compound of formula (1) in a form selected from the group consisting of an intravenous injection, an intramuscular injection, a suppository, an inhalant, a transdermal preparation, an eye drop, and a nasal drop.

21. The method according to claim 16, wherein said one or more pharmaceutically acceptable additives are selected from the group consisting of a pharmaceutically acceptable excipient, a binder, an extender, a disintegrant, a surfactant, a lubricant, a dispersant, a buffering agent, a preservative, a corrigent, a perfume, a coating material, a carrier, and a diluent.

22. The method according to claim 16, wherein said effective amount ranges from 0.01 to 1000 mg/day.

23. The method according to claim 16, wherein said effective amount ranges from 0.1 to 100 mg/day.
